(19) United States
(12) Patent Application Publication
(10) Pub. No.: US 2009/0169420 A1
(43) Pub. Date: Jul. 2, 2009

(54) Time controlled scenting system

(76) Inventors: David John Pollizi, Chardon, OH (US); Don Billy Kincaid, JR., Mentor, OH (US)

Correspondence Address: David J. Pollizi, 9830 Campton Ridge Dr., Chardon, OH, 44024, US

(21) Appl. No.: 12/006,038

(22) Filed: Dec. 31, 2007

(51) Int. Cl. A61L 9/14

(52) U.S. Cl. 422/4; 422/123

(57) ABSTRACT

An automatic fragrance dispensing system used for distributing fragrances and/or anti-microbial solutions throughout a building using the HVAC system. The system includes a printed circuit board which controls the duration of the misting levels, time sequences, and instant misting operations. A dispensing nozzle to atomizing the solutions, an air flow switch to detect air flow when used with the HVAC system, a pump, a reservoir to hold the solution, a conductivity probe for detecting the level of solution in the reservoir, a keyed air flow switch for changing the operation of the fragrance system in order to dispense the solution of choice with air flow or no air flow. The design of this system is to be user friendly and fully adjustable to suit every consumers needs.

### FIELD OF THE INVENTION

The present invention is generally related to an air scenting system, and more particularly to a system that injects fragrances into a forced air system using a time controlled misting apparatus.

### BACKGROUND OF THE INVENTION

Controlling the quality of ambient indoor air is a concern to many homeowners and business owners. Offensive odors adversely affect the quality of the indoor air. Accordingly, these homeowners and business owners use several techniques and devices in an attempt to control these offensive odors. Some such devices are hand held and must be manually operated, thus drawing human resources away from other tasks. Other devices are very expensive to operate and very anesthetically pleasing to view. Still other devices are not effective in sustaining control over the offensive odors. Finally, some devices are incapable of controlling the offensive odors in anything other than a small space, while others devices create an offensive odor to their own.

In other indoor situations, it is simply desirable to improve the surroundings by dispensing selected fragrances at selected times. Such dispensing devices are preferably automated so that they will operate without active human intervention. Many of these systems, however, are expensive to install and operate, are ineffective in dispensing fragrances evenly and throughout large spaces, and are difficult to operate.

Such techniques and devices not only attempt to mask these offensive odors, but also actually attempt to exert a physiological and psychological effect on the individuals present in the area. Previously, this concept was known empirically as “aroma therapy”. More recently, from research in the area of concomitant negative variation (CNV) of human brain waves, it has been verified that certain aromatic substances have calming, or conversely stimulating physiological effects. For example, it has been shown that when lemon, or similar fragrances are dispensed in the air conditioning air of conference rooms, key-punch areas, and the like, that an increase in efficiency occurs and that loss of efficiency through fatigue is diminished. Similarly, rosemary and related substances have been shown to have a calming effect.

Certain such fragrances injection systems use a large amount of water to disperse their fragrances. Many of these systems are located in dark, cool locations that are ripe for producing mold, bacteria, and/or fungus. Additionally, mold, bacteria, and fungus are often very prevalent in areas where such fragrance injection systems are located. Clearly, the propagation of such mold, bacteria, and/or fungus is very detrimental.

Accordingly, there is a need for a system that injects fragrances into a room or area through something such as a forced air HVAC system. There is also a need for such a system using a time controlled misting apparatus to inject such fragrances. There is a need to provide a fragrance injection system that includes certain fungicides, anti-bacterial solutions, and anti-microbial agents with the fragrances to reduce or eliminate the propagation of mold, bacteria, and fungus. Finally, there is a need for a system that provides certain fragrances that may have certain physiological and psychological influences on people.

Additional information will be set forth in the description which follows, and in part will be obvious from the description, or may be learned by practice of the invention.

### DESCRIPTION OF THE DRAWINGS

Operation of the invention may be better understood by reference to the following detailed description taken in connection with the following illustrations, wherein:

FIG. 1 is a diagrammatical view of an embodiment of a time controlled scenting system; and

FIG. 2 is a diagrammatical view of and alternative embodiment of the time controlled scenting system; and

FIG. 3 is a exploded view of an embodiment of a time controlled scenting system; and

FIG. 4 is a exploded view of an alternative embodiment of the time controlled scenting system; and

### DETAILED DESCRIPTION

While the present invention is described with reference to the embodiments described herein, it should be clear that the present invention should not be limited to such embodiments. Therefore, the description of the embodiments herein is illustrative of the present invention and should not limit the scope of the invention as claimed.

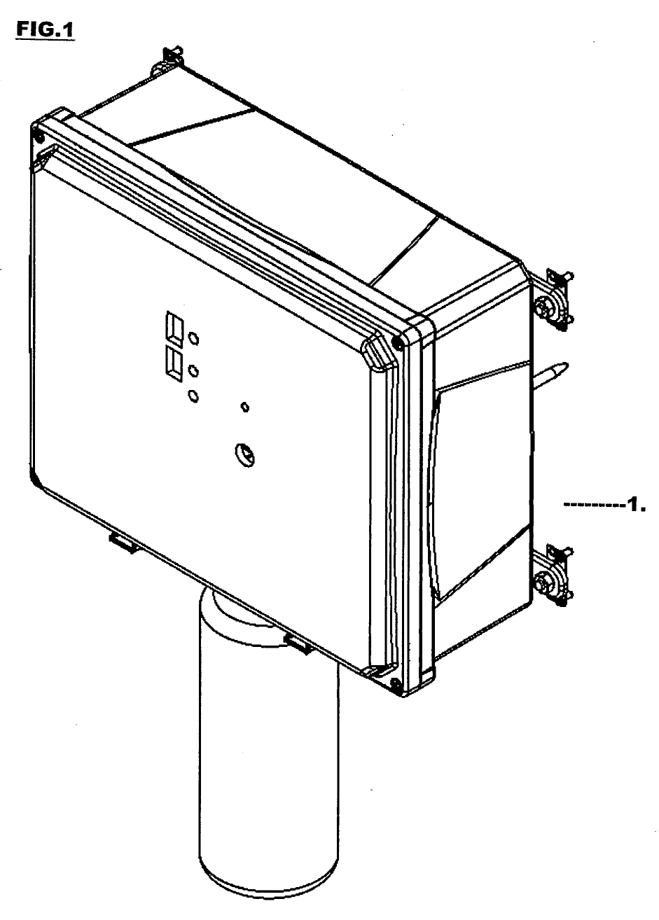

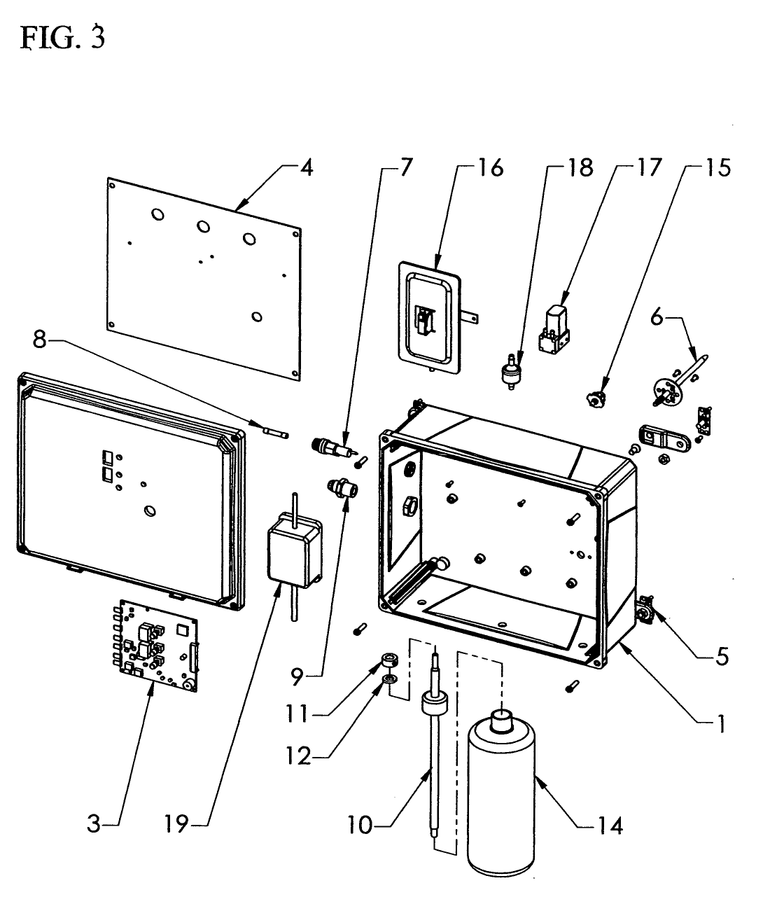

Reference will now be made in detail to the embodiments of the invention as illustrated in the accompanying figures. Shown in FIG. 1 is a time controlled scenting system 1. The system 1 automatically injects a fragrance into a room or area. This can be accomplished through a forced air HVAC system. As shown in FIG. 3, the time controlled scenting system 1 includes a display/operation circuit board panel 3, a power supply 19, a pump 17, a misting nozzle 15, a fragrance container 14, conductivity probe 10, and airflow probe 6, airflow switch 16, back flow check valve 18 mounting brackets 5.

The time controlled scenting system 1 can be attached anywhere in the business or home of the user. For example, the time controlled scenting system 1 can be mounted on or next to the HVAC system or in any other easily accessible area the user chooses. Additionally, the display/operation circuit board panel 3 can be integrated with the thermostat, with a home/business computerized maintenance system, or even integrated with a standard computer. Regardless of the time controlled scenting system 1 location or its integration with another system, it can be operated remotely using a remote control, a PDA, a cell phone, a computer, or any other peripheral device. If the display/operator circuit board panel 3 is not integrated with another system, it will have some sort of display portion and a portion for data entry for control of the system 1. For example, the display/operator circuit board panel 3 may include an LED bar graph with a touch pad. Alternatively, it could include a crystal screen with a touch pad, or even a display/touch screen. Regardless of the display/operator circuit board panel 3 used, it will include a warning system that notifies a user when the fragrance container 14 is low or when the system 1 is not operating properly. This warning system may include a warning LED light and an audible buzzer. If the system 1 includes an audible buzzer, the user may turn the audible buzzer off if so desired until the problem is resolved.

The display/operation circuit board 3 can be any sort of printed circuit board, or other such device. Connected with the power supply 19. This power supply 19 can be a power cord that supplies power to the entire system 1 that is plugged into or hardwired with a home or business's electrical system. The power supply 19 may also include a 12-volt AC transformer so as to power a 12-volt DC pump. Alternatively, the power supply could come from some sort of battery (not shown), or any electrical generation/transmission device.

The display/operation circuit board 3 is also operatively connected with a pump 17. The pump 17 can be any sort of liquid pump, such as the 12-volt DC pump mentioned above. Power is supplied through the display/operation circuit board 3 to the pump 17 to operate it. Alternatively, the pump 17 could have a separate power supply. The pump 17 is then operatively connected with the fragrance container 14 and misting nozzle 15 to pump the selected fragrance from the container 14 through the misting nozzle 15 and into the air. The pump 17 is connected with the fragrance container 14 and misting nozzle 15 through tubing capable of moving liquid. The tubing can be any sort of tubing such as stainless steel tubing or plastic tubing. The fragrance container 14 can be any sort of container of any size that is capable of holding liquid, e.g., it can be a 32 oz plastic bottle. The fragrance container 14 can include a top that is openable and re-closeable so that once it is empty additional liquid fragrance can be added thereto. In this embodiment, the misting nozzle 15 is connected with the fragrance container 14 through the pump 17. Alternatively, the fragrance container may be sealed at its top. In this embodiment, the misting nozzle 15 is connected therewith through the pump 17.

The conductivity probe 10 is positioned within the fragrance container 14. It is positioned at or near the bottom of the fragrance container 14 so that when the liquid fragrance is getting low, the conductivity probe 10 will sense such. The conductivity probe 10 is operatively connected with the display/operation circuit board panel 3 so as to be able to send a signal to the display/operation circuit board panel 3 that the fragrance container 14 is nearly empty or empty. The conductivity probe 10 works by detecting the conductivity of its surroundings (liquid is conductive) and when such conductivity is broken, the user knows that liquid fragrance has dropped below a predetermined level. The conductivity probe 10 uses a siphoning tube to detect the level of the liquid. The user is then notified of this condition through the display/operator circuit board panel 3.

The airflow probe switch 16 is operatively connected with the display/operator circuit board 3. The airflow probe switch 16 detects whether there is an air flow so that when the system 1 mists the fragrance there is sufficient air flow to carry the fragrance out into the chosen room or area. In particular, of the system 1 is placed in an HVAC system, the airflow probe switch 16 detects whether the HVAC is in an operational state pushing air from it. If is, the airflow probe switch 16 will detect such. This is described in more detail below. Alternatively, the key switch 9 is operatively connected with the display/operator circuit board panel 3. The key switch can bypass the airflow probe switch 16 such that regardless of the flow of air, the system 1 can mist the fragrance. A user can actuate the key switch and the system 1 will promptly mist the fragrance. Alternatively, a user can set the key switch such that every hour the system 1 will mist regardless of the detection of the flow of air. As explained in more detail below, misting with some fungicidal, anti-bacterial, and/or anti-microbial agent permits such agent to settle on the air conditioner coil (or any other location so designated) so as to disperse the fungicidal, anti-bacterial, and/or anti-microbial agent.

The power supply 19 can be a 12-volt AC wall transformer, converted to 12V DC through the display/operator circuit board 3, which runs the pump 17 (or pumps) that is pressurizing water-based fragrance through the misting nozzle 15 attached to the end of it. The system 1 uses the conductivity probe 10 and additional circuitry. The display/operation circuit board panel 3 will have a fluid monitor that has a low-level warning light and audio signal. It will automatically shut down if fluid levels drop too low so the pump 17 will not run dry. The fragrance container 14 can be a plastic bottle approximately 32 oz in capacity. The fragrance container 14 will tread on and off for easy installation and has the conductivity probe 10 and a sensing circuitry probe utilized for siphoning the fluid into the pump 17.

The entire system, excluding the power supply 19, may be enclosed in a case making it substantially self contained. The case can be made of any material. However, a hard plastic works well and can be mounted to the side of the furnace plenum with mounting brackets 5. There will also be two to four holes drilled into the furnace plenum to accommodate the airflow probe 6 and misting nozzle 15.

Further, the system 1 can use fragrances that include a fungicidal, anti-bacterial, and/or anti-microbial agent to prevent the propagation of fungus, bacteria, and/or mold. When the system 1 mists the fragrance containing such agents, the spread of such bacteria, fungus, and/or mold is substantially prevented. This is especially useful because of the typical location of the system 1, in the HVAC/furnace plenum. This is a dark moist place that is ripe for the propagation of fungus, mod, and bacteria. Additionally, the fragrances are liquid and this adds to the wetness of the area, which also increases the likelihood of growth of fungus, mold, and/or bacteria. These agents in the fragrances can also even prevent and reduce such fungus, mold, and/or bacteria already present in the HVAC system, rooms, and/or areas of use. As previously explained, a user can actuate the key switch 9 to mist a fungicidal, anti-bacterial, and/or anti-microbial agent regardless of the flow of air or the detection thereof. This allows such agents to be sprayed into the airflow system (HVAC system) to prevent the propagation of fungus, mold, and/or bacteria in the airflow system. Additionally, it can even eliminate the existence of such fungus, mold, and/or bacteria that may already be present in the airflow system.

In addition to these agents, the fragrances can be selected to stimulate concomitant negative variations (CNV) of human brain waves. This can exert a physiological and psychological effect of the individuals present in the area. For example, such fragrances can have a calming, conversely, stimulating physiological effects. For example, it has been shown that when lemon, or similar fragrances are dispersed in the air conditioning air of conference rooms, key-punch areas, and the like, that an increase in efficiency occurs and that loss of efficiency through fatigue is diminished. Similarly, rosemary and related substances have been shown to have a calming effect. These fragrances can be added to the system 1 to exert certain influences on the individuals exposed to such fragrances.

The system 1 operates as follows. A user enters a first timed sequence into the display/operation circuit board panel 3. This first time sequence is in cycles, ranging from 1-8 hours adjusted by a small push button tied to an illuminated numerical read-out indicator on the display/operation circuit board panel 3. This timed controller has continuous power so it can maintain its clock. When the desired time has elapsed, the controller then waits for a signal to send power to energize the second sequence timer. The airflow probe switch 16 provides the signal. When the HVAC system fan is activated, the airflow probe switch 16 detects the flow of air and then sends a signal to send power to energize the second sequence timer.

The second timed sequence is in seconds, ranging from 4-12 seconds, also using a small push button with an illuminated numerical read-out indicator on the display/operator circuit board panel 3. The second timer is then energized to power the pump 17 that emits a fragrance mist into the HVAC system. The first controller resets and starts the procedure over. The system 1 will also have an instant mist/reset button on the display/operation circuit board panel 3 such that upon installation of a new fragrance container 14 or when instant misting is desired, the user merely actuates this button. Upon activation thereof, is the airflow probe switch 16 detects air flow, the system 1 will become active and will release the desired mist of fragrance. If, on the other hand, the airflow probe switch 16 does not detect airflow, the system 1 will not release the mist. The system 1 will remain in the ready state until the airflow probe switch 16 detects the air and activates the system 1.

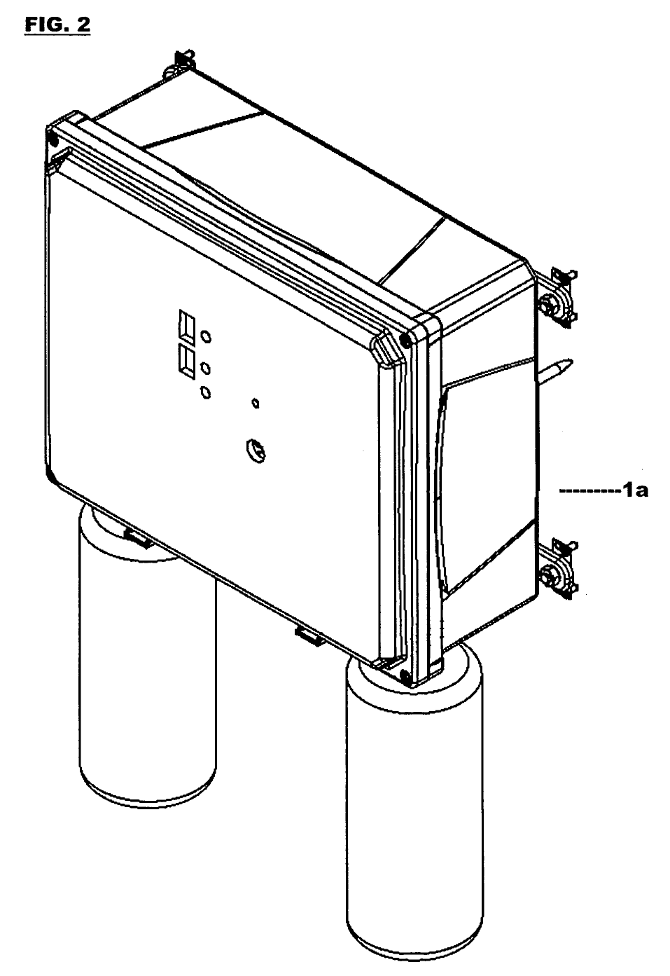

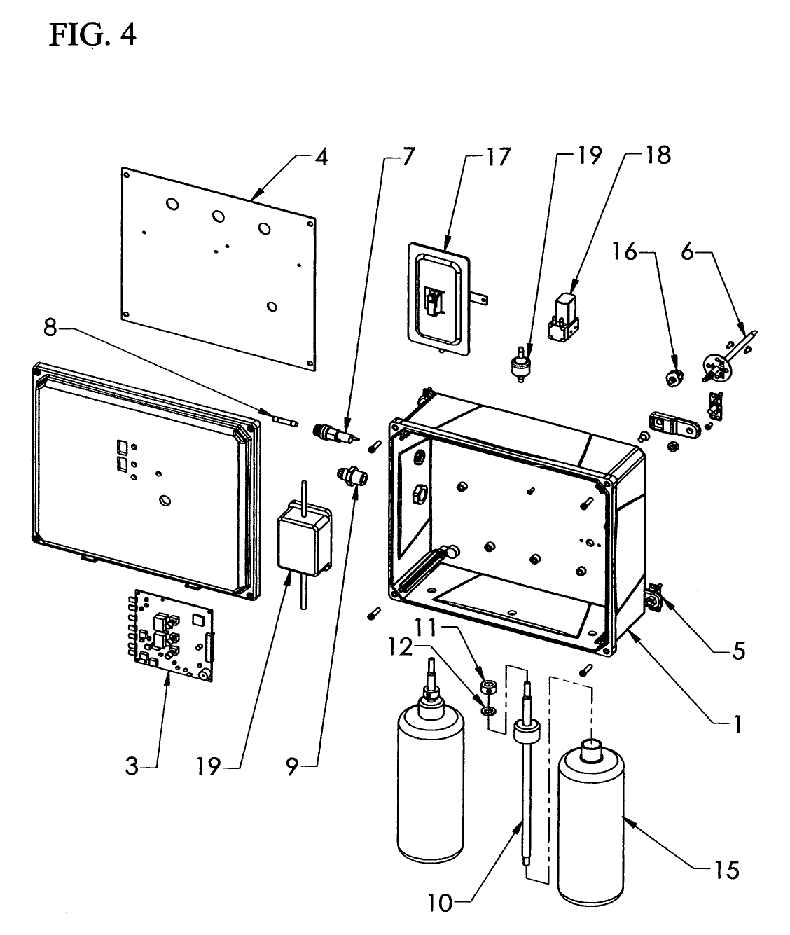

Alternatively, as shown in FIG. 2 and FIG. 4, the system 1 can include two fragrance containers 14, and two conductivity probes. The operation of this system is the same previously explained, however the two fragrances in the two fragrance containers 14 are evenly mixed when misted. The user can add two separate fragrances, e.g., cinnamon and vanilla, so as to create a certain fragrance, e.g., cinnamon buns. Any combination of fragrance can be used; it is up to the user's discretion.

The invention has been described above and, obviously, modifications and alterations will occur to others upon a reading and understanding of this specification.

Having thus described the invention, I claim:

1. A scenting system as substantially shown and described.

2. A method of expelling a fragrance as substantially shown and described.

3. A conductivity probe used to siphon and determine operating modes of the scenting system.

4. A key switch that bypass the airflow probe switch such, that regardless of the flow of air, allowing the system to mist a fungicidal, anti-bacterial, and/or anti-microbial agent in to the HVAC system in order to help prevent the growth of mold and bacteria.

5. A instant mist/reset button when instant misting is desired, the user merely actuates this button. Upon activation thereof, the system will become active and will release the desired mist of fragrance.
